(19) United States
(12) Patent Application Publication
(10) Pub. No.: US 2020/0033332 A1
(43) Pub. Date: Jan. 30, 2020

(54) THIOLATED AROMATIC BLOCKING STRUCTURES FOR EAB BIOSENSORS

(71) Applicant: Eccrine Systems, Inc., Cincinnati, OH (US)

(72) Inventors: Jacob A. Bertrand, Norwood, OH (US); Brian Hanley, Cincinnati, OH (US); Leila Safazadeh Haghighi, Cincinnati, OH (US); Michael Brothers, Lebanon, OH (US)

(73) Assignee: Eccrine Systems, Inc., Cincinnati, OH (US)

(21) Appl. No.: 16/469,358

(22) PCT Filed: Dec. 13, 2017
(86) PCT No.: PCT/US17/66069

§ 371 Date: Jun. 13, 2019

Related U.S. Application Data
(60) Provisional application No. 62/433,368, filed on Dec. 13, 2016.

(51) Int. Cl. G01N 33/543; G01N 33/53

(52) U.S. Cl. CPC G01N 33/5438; G01N 33/5308; C12N 15/115; G01N 2410/00; G01N 2610/00

(57) ABSTRACT

The present invention provides self-assembled monolayers (SAM) configured for use with electrochemical aptamer-based biosensors, which allow sensing devices to detect very low concentrations of target analytes in a biofluid sample. Embodiments of the disclosed invention include SAMs with improved long-term stability in sweat through persistent thiolate bonds between the sensor electrode and disclosed blocker groups, or between the sensor electrode and aptamer sensing elements via disclosed binder molecules. Embodiments of the invention also include blocker groups configured to form densely packed and persistent SAMs on sensor electrodes.

### CROSS-REFERENCE TO RELATED APPLICATIONS

This application claims priority to PCT/US17/66069, filed Dec. 13, 2017, and U.S. Provisional Application No. 62/433,368, filed Dec. 13, 2016, the disclosures of which are hereby incorporated herein by reference in their entirety.

### BACKGROUND OF THE INVENTION

Despite the many ergonomic advantages of sweat for biosensing applications, particularly by wearable devices, sweat remains underutilized compared to blood, urine, and saliva. Upon closer comparison to other non-invasive biofluids, the advantages may even extend beyond ergonomics: sweat may provide superior analyte information. Sweat has many of the same analytes and analyte concentrations found in blood and interstitial fluid.

A number of challenges, however, have historically kept sweat from occupying its place among the preferred clinical biofluids. These challenges include very low sample volumes (nL to μL), unknown concentrations due to evaporation, filtration and dilution of large analytes, mixing of old and new sweat, and the potential for contamination from the skin surface. More recently, rapid progress in wearable sweat sampling and sensing devices has resolved several of the historical challenges. However, this recent progress has also been limited to high concentration analytes (μM to mM) sampled at high sweat rates (>1 nL/min/gland) found in, for example, athletic applications. Progress will be much more challenging as sweat biosensing moves towards detection of large, low concentration analytes (nM to pM and lower).

In particular, many known sensor technologies for detecting larger molecules are ill-suited for use in wearable sweat sensing, which requires sensors that permit continuous use on a wearer's skin. This means that sensor modalities that require complex microfluidic manipulation, the addition of reagents, the use of limited shelf-life components, such as antibodies, or sensors that are designed for a single use, will be unsuitable for sweat sensing. By contrast, electrochemical aptamer-based (“EAB”) sensor technology, such as the multiple-capture EAB sensors disclosed in U.S. Pat. Nos. 7,803,542, and 8,003,374, presents a stable, reliable bioelectric sensor that is sensitive to the target analyte in sweat. Similarly, U.S. Provisional Application No. 62/523,835, filed Jun. 23, 2017 and incorporated by reference herein in its entirety, presents a docked aptamer EAB sensor also for use with the disclosed invention.

One difficulty for using EAB sensors in sweat sensing devices is the relatively poor long-term stability of EAB sensing elements when exposed to the sweat medium. One cause of this instability is the tendency of thiolate bonds (such as are commonly used to attach aptamer sensing elements and blocker groups to gold electrodes) to degrade, especially in the presence of interrogation currents. Accordingly, as disclosed herein, a solution is provided that improves the stability of thiolate bonds between electrodes and attached aptamer sensing elements and blocker groups.

### SUMMARY OF THE INVENTION

The present invention provides self-assembled monolayers (SAM) configured for use with electrochemical aptamer-based biosensors, which allow sensing devices to detect very low concentrations of target analytes in a biofluid sample. Embodiments of the disclosed invention include SAMs with improved long-term stability in sweat through persistent thiolate bonds between the sensor electrode and disclosed blocker groups, or between the sensor electrode and aptamer sensing elements via disclosed binder molecules. Embodiments of the invention also include blocker groups configured to form densely packed and persistent SAMs on sensor electrodes.

### BRIEF DESCRIPTION OF THE DRAWINGS

The objects and advantages of the present invention will be further appreciated in light of the following detailed descriptions and drawings in which:

FIG. 1 represents an aptamer sensing element of a previously-disclosed EAB sensor.

FIG. 2 depicts a molecule for use in an embodiment of the disclosed invention.

FIG. 3 depicts an embodiment of the disclosed invention.

FIG. 4 depicts a chemical reaction relevant to an embodiment of the disclosed invention.

FIG. 5 depicts a chemical reaction relevant to an embodiment of the disclosed invention.

FIGS. 6A & 6B depict an example synthesis reaction for an embodiment of the disclosed invention.

FIG. 7 depicts an example molecule for use in an embodiment of the disclosed invention.

FIG. 8 depicts an example molecule for use in an embodiment of the disclosed invention.

FIG. 9 depicts an example molecule for use in an embodiment of the disclosed invention.

FIG. 10 depicts an example molecule for use in an embodiment of the disclosed invention.

FIG. 11 depicts an example molecule for use in an embodiment of the disclosed invention.

FIG. 12 depicts an example molecule for use in an embodiment of the disclosed invention.

FIGS. 13A, 13B, & 13C depict an example molecule for use in an embodiment of the disclosed invention.

### DEFINITIONS

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva. For sweat sensing applications as generally discussed herein, biofluid has a narrower meaning, namely, a fluid that is comprised mainly of interstitial fluid or sweat as it emerges from the skin.

“Continuous monitoring” means the capability of a device to provide at least one measurement of sweat determined by a continuous or multiple collection and sensing of that measurement or to provide a plurality of measurements of sweat over time.

“Chronological assurance” means the sampling rate or sampling interval that assures measurement(s) of analytes in sweat in terms of the rate at which measurements can be made of new sweat analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

“Sweat stimulation” is the direct or indirect causing of sweat generation by any external stimulus, the external stimulus being applied to stimulate sweat. One example of sweat stimulation is the administration of a sweat stimulant such as pilocarpine or carbachol. Going for a jog, which stimulates sweat, is only sweat stimulation if the subject jogging is jogging in order to stimulate sweat.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Measured” can imply an exact or precise quantitative measurement and can include broader meanings such as, for example, measuring a relative amount of change of something. Measured can also imply a binary measurement, such as ‘yes’ or ‘no’ type measurements.

“Analyte” means a substance, molecule, ion, or other material that is measured by a sweat sensing device.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence of the target analyte. Such sensors can be in the forms disclosed in U.S. Pat. Nos. 7,803,542 and 8,003,374 (the “Multiple-capture Aptamer Sensor” (MCAS)), or in U.S. Provisional Application No. 62/523,835 (the “Docked Aptamer Sensor” (DAS)).

“Analyte capture complex” means an aptamer, oligomer, or other suitable molecules or complexes, such as proteins, polymers, molecularly imprinted polymers, polypeptides, and glycans, that experience a conformation change in the presence of a target analyte, and are capable of being used in an analyte-specific sensor. Such molecules or complexes can be modified by the addition of one or more primer sections comprised of nucleotide bases.

“Aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

“Biorecognition element” means an aptamer or other molecule that interacts with a target analyte molecule and can be functionalized as part of a biosensor, including without limitation, proteins, polymers, molecularly imprinted polymers, polypeptides, and glycans.

“Aptamer sensing element” means an analyte capture complex that is functionalized to operate in conjunction with an electrode to detect the presence of a target analyte. Such functionalization may include tagging the aptamer with a redoxable moiety, or attaching thiol binding molecules, docking structures, or other components to the aptamer. Multiple aptamer sensing elements functionalized on an electrode comprise an EAB sensor.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

“Self-assembled monolayer” (SAM) means a layer of molecules that adhere to an EAB electrode through self-limiting binding reactions, i.e., each open surface molecule will react with only one introduced gaseous molecule. Therefore, individual layers can be deposited with great precision, allowing several layers, e.g., 3 to 50 layers, each of which is about 1 Å thick, to be built up on the electrode to form a blocking surface.

### DETAILED DESCRIPTION OF THE INVENTION

Embodiments of the disclosed invention apply at least to any type of sweat sensing device that measures at least one analyte in sweat, interstitial fluid, or other biofluid. Further, embodiments of the disclosed invention apply to sensing devices which measure samples at chronologically assured sampling rates or intervals. Embodiments of the disclosed invention apply to sensing devices which can take various forms including patches, bands, straps, portions of clothing, wearables, or any suitable mechanism that reliably brings sampling and sensing technology into intimate proximity with sweat sample as it is transported to the skin surface. While some embodiments of the disclosed invention utilize adhesives to hold the device near the skin, devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sensing devices with more sub-components needed for use of the device in various applications, which are obvious (such as a battery), and for purposes of brevity and of greater focus on inventive aspects, such components are not explicitly shown in the diagrams or described in the embodiments of the disclosed invention.

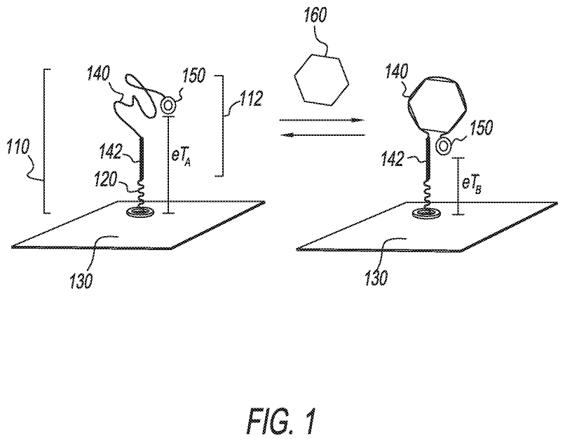

As disclosed in PCT/US17/23399, filed Mar. 21, 2017, incorporated by reference in its entirety herein, EAB sensors for use in continuous sweat sensing are configured to provide stable sensor responses over time in the presence of a mostly continuous or prolonged flow of sweat sample. For example, the multiple-capture EAB sensor includes a plurality of individual aptamer sensing elements, as depicted in FIG. 1, which can repeatedly detect the presence of a molecular target by capturing and releasing target analytes as they interact with the aptamer. The sensing element includes an analyte capture complex 140 that has a first end covalently bonded to a primer 142. The primer 142 is bonded to a sulfur molecule (thiol) 120, which is in turn covalently bonded to a gold electrode base 130. In other embodiments (not shown), the aptamer may be bound to the electrode by means of an ethylenediaminetetraacetic acid (EDTA) strain, to improve adhesion in difficult sensing environments, such as sweat biofluid. The sensing element further includes a redox moiety 150 that may be covalently bonded to the aptamer 140 or bound to it by a linking section. In the absence of the target analyte, the aptamer 140 is in a first configuration, and the redox moiety 150 is in a first position relative to the electrode 130. When the sweat sensing device interrogates the sensing element using square wave voltammetry (SWV), the sensing element produces a first electrical signal, eT_{A}.

The aptamer 140 is selected to specifically interact with a target analyte 160, so that when the aptamer captures a target analyte molecule, the aptamer undergoes a conformation change that partially disrupts the first configuration, and forms a second configuration. The capture of the target analyte 160 accordingly moves the redox moiety 150 into a second position relative to the electrode 130. Now when the sweat sensing device interrogates the sensing element, the sensing element produces a second electrical signal eT_{B} that is distinguishable from the first electrical signal. After an interval of nanoseconds, milliseconds, seconds or longer, (the “recovery interval”), the aptamer 140 releases the target analyte 160, and the aptamer returns to the first configuration, which will produce the corresponding first electrical signal when the sensing element is interrogated.

To enable the aptamer sensing element, the electrode surface is further prepared by adding passivating reagents to create blocker groups that prevent non-specific adsorption on the surface. If not prevented, non-specific molecules that foul the electrode surface will cause the sensing element to register a weaker signal in the presence of an analyte, thus raising the lower limit of detection for the sensor. Electrode passivation is accomplished by treating the surface with hydroxyl-terminated alkane thiols, which adhere to the electrode and sterically block the (otherwise) uncovered area. See Lai, Plaxco, et al. 2007. The passivating reagents commonly used to form the blocker groups are short-chain monothiols such as 6-mercapto, 1-hexanol (MCH), and 3-mercaptopropionic acid (MPA), which form a self-assembled monolayer (SAM) adsorbate through spontaneous adsorption from the solution onto the gold surface.

A major limitation of EAB sensors is their relatively poor long-term stability in the presence of sweat biofluid. While EAB sensor technology is relatively well known in the art, few practitioners have systematically studied the stability of this type of sensor in sweat, or how stability may be improved. See Phares, White, et al. 2009. The disclosed invention, therefore, provides a solution to improve the long-term stability of EAB sensors in biofluids such as sweat.

One reason for the relatively poor stability of EAB sensors is their dependence on the self-assembly of thiolated aptamers and blocker groups. While such self-assembled monolayers are easy to fabricate, they exhibit low long-term stability due to gradual desorption of thiol moieties from the electrode surface. As a consequence, both the aptamer sensing elements and the thiol blocker groups tend to dissociate from the electrode, rendering the EAB sensor ineffective, or less effective, over time. This desorption is likely caused by transient oxidization events and the application of reduction potentials to interrogate the sensors, both of which weaken the gold-thiol bonds. See Poirier, Tarlov, et al., 1994; Flynn, Tran, et al., 2003. Evidence indicates that the desorption of thiols from the gold occurs according to either of the following reactions:

2RS—Au→2RSH+2Au→RSSR+2Au equation 1:

RS—Au+H₂O+O₃→RSO₃H+HO—Au equation 2:

According to equation 1, thiols as disulfides can form stable but reversible complexes that resist immediate reattachment to the electrode surface. Similarly, according to equation 2, thiols as sulfonates will not adsorb to the gold surface, and thus can be easily removed. In addition to such preferential chemical pathways, it has also been shown that SAMs on gold surfaces become less stable at high temperatures (>40° C.) and high salt concentrations (>0.3 M). See Li, Jin, et al., 2002.

EAB sensor stability would theoretically be improved by using longer-chain thiols to tether the aptamer sensing elements, and to serve as blocker groups, see Ulman, A., 1996, however, using longer chain thiols will diminish the electron transfer (and hence the signal) produced by the aptamer sensing element upon analyte capture. As disclosed herein, therefore, an embodiment of the invention instead uses multiple thiols, e.g., trihexylthiol, to anchor the aptamer sensing elements and blocker groups to the electrode. See Li, Jin, et al., 2002; Sakata, Maruyama, et al., 2007; Phares, N., et al., 2009. Aptamer sensing elements anchored with flexible trihexylthiol molecules exhibit enhanced stability, retaining 75% of their original signal and maintaining excellent signaling properties after fifty days of storage in buffer. The disclosed approach also preserves the EAB sensor's stability without diminishing electron transfer or otherwise degrading sensor performance during use.

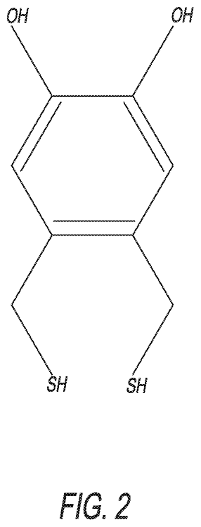

Another important factor in the stability of aptamer based sensors is the physical structure of the alkane thiol blocker groups. As shown in literature, having multiple attachment sites available significantly improves the stability of thiolated adsorbate films. Therefore, in an embodiment of the disclosed invention, conventional monothiol blocker groups such as MCH are replaced with molecules bearing multiple thiols. Specifically, one alternative blocking molecule is 4,5-bis(sulfanylmethyl)benzene-1,2-diol (including its isomers), see FIG. 2, as well as analogous molecules that possess the following properties: 1) a pair of ortho-methane thiol groups; 2) an aromatic ring consisting of 6 carbons; and 3) 1-4 phenolic hydroxyls (regardless of position). Such molecules may also be used to tether aptamer sensing elements to an electrode.

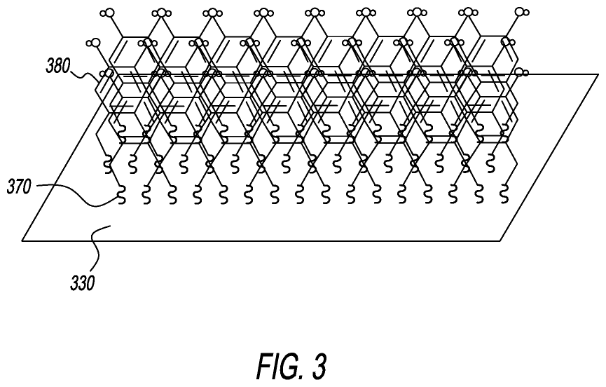

The adsorbate molecules of aromatic dithiol-based SAMs form a closely-packed and ordered structure on the gold electrode substrate, assuming a 2×2 overlayer structure. The ordered structure is stabilized through multiple gold-sulfur interactions, intermolecular van der Waals bonds, and hydrogen bonds, both within the monolayer and between the aromatic moieties. With reference to FIG. 3, a portion of an EAB biosensor is depicted that includes the disclosed polythiol binders and blocker groups. The biosensor includes an electrode 320, with a plurality of binder molecules 370 that attach a plurality of blocker groups 380 to the electrode. The blocker groups interact with each other to form a densely-packed, orderly, and stabilized SAM on the electrode, which enhances the function of the plurality of aptamer sensing elements (not shown), that may be attached to the electrode or to the SAM.

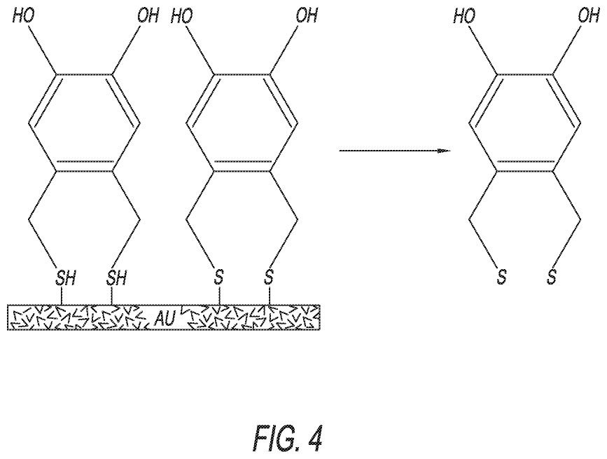

Among the characteristics of such aromatic dithiol blocker groups that promise to improve long-term EAB sensor stability are the following: First, the two proximal thiol groups in 4,5-bis(sulfanylmethyl)benzene-1,2-diol provide two bonding moieties for attachment to the gold surface. Multiple sulfur ligands are known to promote the stability of sulfur-tethered films, and in addition, the entropy-driven chelate effect tends to improve the stability of homogeneous organometallic complexes, like dithiol groups. Second, π-π stacking interactions should provide additional enthalpic and entropic contributions to blocker group stability even without tethered molecules. Third, the free phenolic hydroxyls should provide hydrogen bonds that further stabilize the surface, and create an amphoteric bilayer. See FIG. 4.

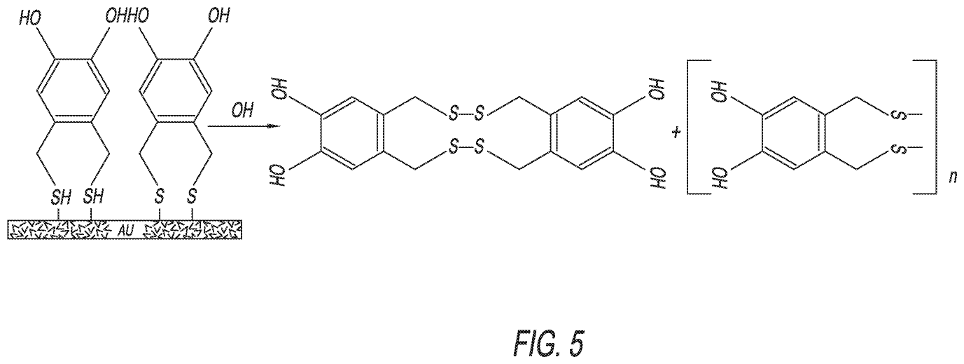

Moreover, desorption pathways involving the formation of intermolecular disulfides are less important for dithiol blocker groups, given that these pathways would require the concurrent desorption of four or more tethered sulfur atoms, which is a low probability event. In addition, such interactions are reversible for dithiol blocker groups. See FIG. 5.

The final benefit of the proposed blocker group molecule is that it is small enough not to degrade electron transfer and aptamer signaling. The smaller size of the proposed blocker molecules allows denser placement on the electrode, and provides less leeway for the molecules to spread out from their attachment point. The use of a small molecule like 4,5-bis(sulfanylmethyl)benzene-1,2-diol has heretofore been overlooked in the art because small molecules are perceived as too unstable for use in EAB sensors despite their desirable properties in relation to electron transfer and signaling. However, utilizing a short, stable SAM with aromatic properties (which improve electrical conductance and reduce resistance) as disclosed, will greatly improve electron transfer characteristics. Similarly, any linker section, regardless of length, that incorporates a conjugated system would have enhanced electron transfer, and thus enhanced signal.

The following example synthesis is generally applicable to dithiol groups as contemplated by the disclosed invention, and can be used for functionalizing the open ring carbons with a number of different functional groups. The example synthesis is broken down into a C—H functionalization step using palladium (II) acetate (Pd(OAc)₂) as the catalyst to acetylate the 4th and 5th carbons on the aromatic ring. Once functionalized using known synthetic methods, the acetate groups could translate into multiple functional groups, but for present purposes, the acetate is only reduced to a hydroxyl group.

### Example Synthesis Step 1

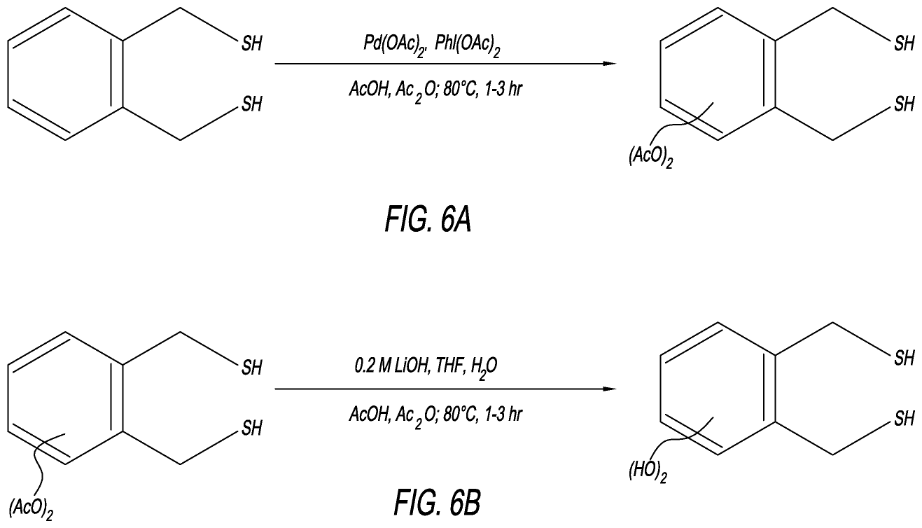

With reference to FIG. 6A, this step is a Pd(OAc)₂ catalyzed acetylation of benzene. The combination of Pd(OAc)₂ as the catalyst and (diacetoxyiodo)benzene (PhI(OAc)₂) as the oxidant functionalizes the C—H bonds in the benzene ring. The (1,2-phenylene) dimethanethiol (1 equiv.), and Pd(OAc)₂ (10 mol. %), PhI(OAc)₂ (2.2 equiv.), are dissolved in acetic acid (AcOH) and acetic anhydride (Ac₂O) (1:1). The resulting mixture is stirred for 1 to 3 hours at 80° C. Purification and separation of the final product is achieved through evaporation of the solvent and column chromatography allowing the product to be isolated. The purified product 4,5-bis(sulfanylmethyl)-1,2-phenylene is confirmed through the use of known analytical methods, including nuclear magnetic resonance imaging, and mass spectroscopy, proton and carbon. This step has an ideal yield of 77%.

### Example Synthesis Step 2

With reference to FIG. 6B, this step is a reduction of the acetate groups using lithium hydroxide (LiOH) in low molarity and at ambient temperature. Add aqueous 0.2 M LiOH to a solution of 4,5-bis(sulfanylmethyl)-1,2-phenylene diacetate (1 equiv.) in tetrahydrofuran (THF), at 25° C., and stir the mixture for 1 to 2 hours. Then, quench the reaction mixture with H₂O (5× the amount of LiOH). The biphasic reaction mixture is then extracted with ethyl acetate (EtOAc), and the combined organic layers are dried using magnesium sulfate (MgSO₄), and then concentrated. Separation and purification with chromatography provides the final product of 4,5-bis(sulfanylmethyl)benzene-1,2-diol. This reaction step has an ideal yield of 92%.

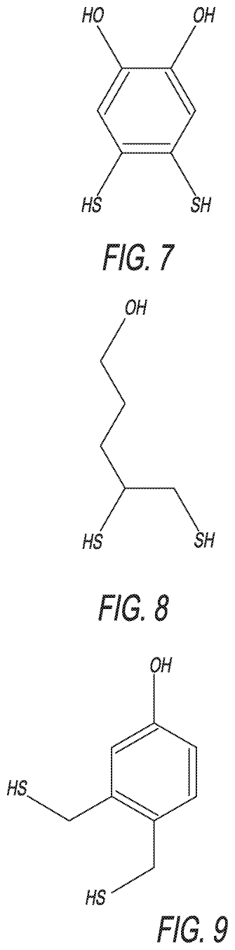

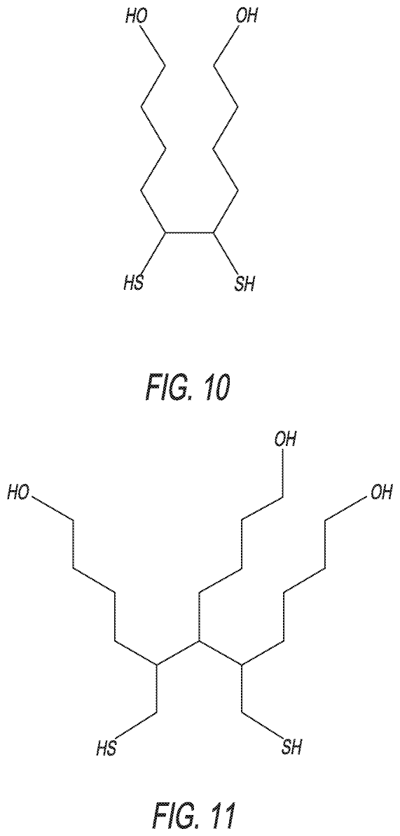

The disclosed enhanced-stability dithiol blocker group/aptamer tether may be alternatively formulated. For example, possible alternative versions include the following: 4,5-bis(sulfanyl)benzene-1,2-diol, see FIG. 7; 4,5-bis(sulfanyl)pentan-1-ol, see FIG. 8; 3,4-bis(sulfanylmethyl)phenol, see FIG. 9; 5,6-bis(sulfanyl)decane-1,10-diol, see FIG. 10; 5,7-bis(sulfanylmethyl)-6-butanolundeca-1,11-diol, see FIG. 11; 1,2-dimercaptopentan-5-ol; and 5,6-dimercaptodecan-1,10-diol.

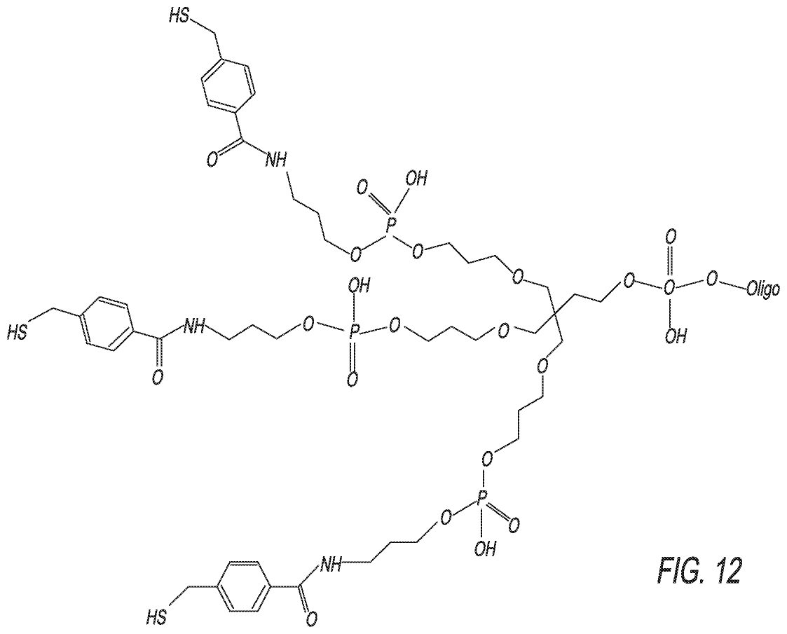

In another alternate embodiment, the blocker group/aptamer tether can comprise a branched trithiol that contains an aromatic moiety, such as a benzyl. See FIG. 12. Several more complex arrangements are also possible. For instance, the molecule may include a plurality of thiols with the aromatic moiety. The molecules can be placed on the electrode surface without further modification to function as blocker groups, or they may be incorporated into the aptamer DNA to function as a tether, using phosphoramidite chemistry, as is familiar to those skilled in the art of oligonucleotide synthesis.

In another alternate embodiment, the disclosed self-assembled monolayer can be attached to electrodes comprised of alternate materials, such as graphite, carbon nanotubes, or other carbon-based materials having a graphene surface characterization. Such techniques previously have been disclosed in U.S. Provisional Application No. 62/559,857, incorporated by reference herein in its entirety. While electrodes constructed from alternate materials may present advantages over gold electrodes for use in biofluid sensing devices, reliably attaching components to the alternate material is a significant problem. Attachment chemistries can vary widely with the properties of the prospective electrode material, and the materials may display additional chemical properties that interfere with the function of EAB sensing elements. Therefore, the adaptable self-assembled monolayers disclosed herein present effective means of attaching layers of molecules to various carbon-based electrode materials, allowing these materials to be used in EAB biosensors.

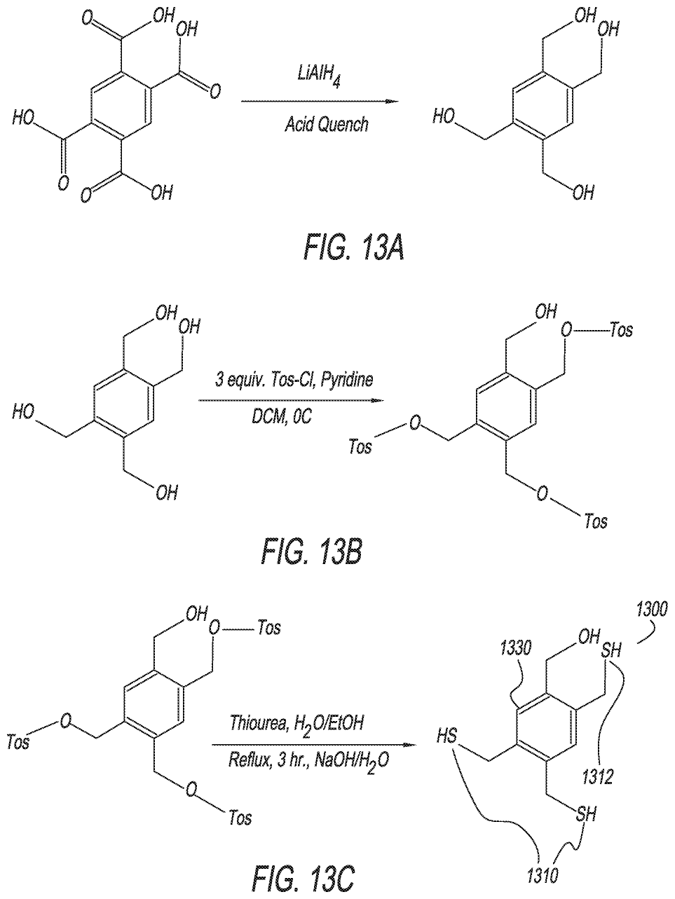

FIGS. 13A-13C depict a series of synthesis reactions for creating a product that, when combined with a plurality of such molecules and functionalized on an EAB sensor electrode surface, serves as an example embodiment of the disclosed invention. With reference to FIG. 13C, the final product, 1-methanol-2,4,5-Benzenetrimethanethiol 1300, can be deposited on an electrode to form a SAM with a number of desirable features. For example, the blocker group 1300 has multiple thiol groups available for forming persistent bonds to an electrode surface 1310 or other components 1312, such as an aptamer sensing element. Also, the aromatic rings 1330 allow individual SAM molecules to interact though π-π stacking. These π-π bonds cause the blocker groups to arrange themselves into a regular and closely-packed surface, creating a dense layer that, in turn will display improved resistance to non-specific fouling of the electrode surface, and improved adhesion of EAB components to the electrode. The relatively small size of the blocker group 1300 also serves to keep the thiol groups closer to the main body of the molecule, which produces a smaller molecule footprint, which also allows the molecules to be packed more densely on the electrode.

The example synthesis depicted in FIGS. 13A-13C is not limited to 1-methanol-2,4,5-Benzenetrimethanethiol, but similar processes can be used to produce SAM components that feature other combinations of alkyl chains, aromatic rings, different functional groups, and additional thiols. SAMs comprised of such substances are also contemplated within the scope of the disclosed invention. For example, SAM components can include functional groups, such as azide groups, that allow click chemistry reactions. Click chemistry reactions can allow robust attachment of a wide variety of compounds, enabling SAM properties to be customized for specific applications. See U.S. Provisional Application No. 62/559,857, filed Sep. 18, 2017, which is hereby incorporated by reference herein in its entirety.

In another embodiment, individual blocker groups are configured to interact with each other through crosslinking reactions. In addition to having thiols to bind the blocker group to the electrode surface, each blocker group also includes thiol attachment points for attaching functional groups for crosslinking the individual blocker groups, or carbon chain branches that include such functional groups. For example, 1-methanol-2,4,5-Benzenetrimethanethiol depicted in FIG. 13C is configured with a polythiol in the 1312 location that allows crosslinking with other blocker groups. Similarly to the action of π-π bonds, crosslinking the individual blocker groups causes the SAM to effectively form a macromolecule, which would display improved resistance to non-specific adsorption, and improved stability through better adherence to the electrode.

For the assembly of SAMs employing crosslinked blocker groups, the crosslinking functional groups would be present but unreactive during SAM self-assembly on the electrode. Once the SAM has been attached to the electrode surface, the crosslinking functional groups could be activated through photon exposure, temperature changes, introduction of a radical species, or other mechanisms known to the those in the field of polymer chemistry. Alternately, the functional groups or other components may be added in multiple steps as required to assemble the desired SAM.

Some examples of suitable functional group chemistries that facilitate such crosslinking include epoxies, polyethers, polyesters, polyurethanes, vinyls, allyl acetate, allyl cyanide, cyano butanes, and acrylates. Examples for monomers that function as the individual blocker groups include, without limitation, styrene, acrylic acid, and vinylpyrrolidone. In other embodiments, blocker groups may be multipurpose, and will include attachment points for crosslinking, in addition to attachment points for functionalizing the SAM by attaching linkers or aptamer sensing elements. Such multifunctional blocker groups will employ orthogonal chemistries so that crosslinking attachment points will not interact with functionalization attachment points.

1. A self-assembled monolayer for an electrochemical aptamer-based (EAB) biosensor, the self-assembled monolayer comprising:
a plurality of binder molecules for attaching a component to a surface of an electrode configured for use in the EAB biosensor, wherein the each of the plurality of binder molecules has a stability characteristic; and
a plurality of blocker groups, wherein each of the plurality of blocker groups include a plurality of thiol attachment points, and wherein the plurality of blocker groups substantially occupy the surface of the electrode, so that non-specific substances are prevented from binding to the surface of the electrode.

2. The self-assembled monolayer of claim 1, wherein the surface is comprised of one of the following: gold (Au), graphene, carbon nanotubes, and graphite.

3. The self-assembled monolayer of claim 1, wherein each of the plurality of binder molecules is comprised of one of the following: trihexylthiol, and ethylenediaminetetraacetic acid.

4. The self-assembled monolayer of claim 1, wherein each of the plurality of binder molecules is comprised of one of the following: a pair of ortho-methane thiol groups; an aromatic ring consisting of 6 carbons; and 1-4 phenolic hydroxyls.

5. The self-assembled monolayer of claim 4, wherein each of the plurality of binder molecules is comprised of one of the following molecules: 4,5-bis(sulfanylmethyl)benzene-1,2-diol; 4,5-bis(sulfanyl)benzene-1,2-diol; 4,5-bis(sulfanyl)pentan-1-ol; 3,4-bis(sulfanylmethyl)phenol; 5,6-bis(sulfanyl)decane-1,10-diol; 5,7-bis(sulfanylmethyl)-6-butanolundeca-1,11-diol; 1,2-dimercaptopentan-5-ol; and 5,6-dimercaptodecan-1,10-diol.

6. The self-assembled monolayer of claim 1, wherein each of the plurality of the binder molecules includes a branched trithiol that includes an aromatic moiety.

7. The self-assembled monolayer of claim 6, wherein the aromatic moiety is a benzyl molecule.

8. The self-assembled monolayer of claim 6, wherein each of the plurality of binder molecules includes a second plurality of attachment points, each comprising a thiol.

9. The self-assembled monolayer of claim 1, wherein the component is an aptamer sensing element, comprising a biorecognition element, a redox moiety, and one or more oligonucleotide linkers.

10. (canceled)

11. The self-assembled monolayer of claim 1, wherein one or more of the plurality of blocker groups is comprises a trihexylthiol.

12. The self-assembled monolayer of claim 1, wherein each of the plurality of blocker groups is comprised of one of the following: a pair of ortho-methane thiol groups; an aromatic ring consisting of 6 carbons; and 1-4 phenolic hydroxyls.

13. The self-assembled monolayer of claim 12, wherein each of the plurality of blocker groups is comprised of one of the following: 4,5-bis(sulfanylmethyl)benzene-1,2-diol; 4,5-bis(sulfanyl)benzene-1,2-diol; 4,5-bis(sulfanyl)pentan-1-ol; 3,4-bis(sulfanylmethyl)phenol; 5,6-bis(sulfanyl)decane-1,10-diol; 5,7-bis(sulfanylmethyl)-6-butanolundeca-1,11-diol; 1,2-dimercaptopentan-5-ol; and 5,6-dimercaptodecan-1,10-diol.

14. The self-assembled monolayer of claim 1, wherein each of the plurality of blocker groups includes a branched trithiol that includes an aromatic moiety comprising a benzyl molecule.

15. (canceled)

16. The self-assembled monolayer of claim 14, wherein each of the plurality of blocker groups includes a second plurality of attachment points each comprising a thiol.

17. The self-assembled monolayer of claim 1, wherein each of the plurality of blocker groups comprises an attachment point configured to attach a component to the blocker group.

18. The self-assembled monolayer of claim 1, wherein each of the plurality of blocker groups is configured to allow π-π stacking with an adjacent blocker group.

19. The self-assembled monolayer of claim 1, wherein each of the plurality of blocker groups includes a group configured to allow an attachment by a click chemistry reaction.

20. The self-assembled monolayer of claim 1, wherein each of the plurality of blocker groups is configured to allow a crosslink chemical reaction with an adjacent blocker group.

21. The self-assembled monolayer of claim 20, wherein each of the plurality of blocker groups further comprises a functional group configured to attach a component.
